(19) United States
(12) Patent Application Publication
(10) Pub. No.: US 2026/0124070 A1
(43) Pub. Date: May 7, 2026

(54) EYE CARE DEVICE

(71) Applicant: Janet Lund, Kelowna (CA)

(72) Inventor: Janet Lund, Kelowna (CA)

(21) Appl. No.: 19/375,887

(22) Filed: Oct. 31, 2025

Related U.S. Application Data
(60) Provisional application No. 63/717,498, filed on Nov. 7, 2024.

(51) Int. Cl. A61F 9/00; A61F 13/38; A61F 13/53

(52) U.S. Cl. CPC A61F 9/00; A61F 13/38; A61F 2013/530014; A61F 2013/530897

(57) ABSTRACT

An eye care device that is operable to absorb tears and similar liquid from an eye wherein the device is provided in a disposable format. The device includes a lower portion integrally formed with an upper portion. The lower portion is planar in manner having a first side and a second side. The upper portion is manufactured from an absorbent lint free material such as but not limited any combination of polyamide, polyester, polypropylene or medical grade cellulose fibers. The upper portion of the present invention includes a bulbously formed middle section that tapers into the upper end of the lower portion. The top section of the upper portion has a precisely formed tip member that reaches the meniscus at the lid margin of the eye without contacting the cornea. The outer surface has disposed thereon a multitude of porous channels that wick tears by capillary action.

### PRIORITY UNDER 35 USC SECTION 119(e) & 37 CFR SECTION 1.78

This nonprovisional application claims priority based upon the following prior United States Provisional Patent Application entitled: Eye Care Device, Application No.: 63/417,498, filed Nov. 7, 2024, in the name of Janet Lund, which is hereby incorporated by reference for all purposes.

### FIELD OF THE INVENTION

The present invention relates generally to eye care, more specifically a device that is held with the fingers of the user wherein the device includes an upper portion manufactured from a combination of materials such as but not limited to polyamide, polyester and polypropylene wherein the upper portion is supported by a handle member and is operable to absorb tears/moisture from the eye and area proximate thereto.

### BACKGROUND

Tearing (epiphora) is the overflow of the eye's watery layer onto the lids, lashes, and skin. It can be physiologic, your eye's normal response to emotion or irritants or pathologic, a sign something isn't draining or lubricating properly. Either way, the lacrimal glands produce fluid that should spread with each blink and then drain through tiny puncta into the nasolacrimal duct. When production overwhelms drainage or drainage is blocked tears spill over. There are many routine causes of tearing. One cause is reflex tearing from irritation such as wind, cold air, smoke, perfumes, cutting onions (sulfur compounds), chlorinated pools, and dust trigger ocular surface nerves. The brain responds by flooding the eye to dilute and wash away the irritant. Another cause is dry eye that paradoxically waters when the tear film's oily layer, mostly from meibomian glands, is poor and the watery layer evaporates too fast. The surface of the eye gets dry, nerves fire, and the lacrimal gland overcompensates with bursts of watery tears which is often worse outdoors or on screens. A further cause is allergic conjunctivitis which is caused by pollen, pet dander, and mites can inflame the conjunctiva. Itching, redness, swelling, and stringy discharge often accompany copious tearing. Still another cause is eye lid margin problems like entropion (inward-turning lid) or ectropion (outward) disrupt tear distribution and drainage. Drainage obstruction can also occur and tears cannot exit if puncta are too small, the canaliculi scarred, or the nasolacrimal duct narrowed or blocked. Individuals often notice one-sided, persistent overflow and recurrent infections. A further cause of tearing are Infections and irritants on the surface of the eye. Examples include viral or bacterial conjunctivitis, contact lens overwear, retained makeup, or sunscreen in the eye all provoke tearing. An additional cause is neurologic and gustatory tearing wherein strong emotions are the classic trigger. Medications and environmental load such as certain eye drops, preservatives, systemic meds, high screen time with infrequent blinking, HVAC airflow, or very low humidity can all contribute. Persistent tearing, pain, light sensitivity, or changes in vision warrant an eye exam; tearing is often benign, but it can also flag treatable problems.

Conventional devices and materials are utilized to wipe off tears and these have their advantages and disadvantages. Facial tissues are widely available, inexpensive, disposable, reasonably absorbent, discrete. However facial tissues shed lint fibers that cling to lashes, lotions/fragrances on the tissue can sting, repetitive wiping abrades thin eyelid skin, bulky boxes aren't portable and facial tissues are not sterile. Toilet paper, napkins, paper towels are utilized by individuals and have several advantages with one being these items are readily available in kitchens/restrooms, have high absorbency. However, these items have rough fibers and embossing that can irritate skin and lids, they have flakes that disintegrate with moisture, paper towels are too abrasive for periocular skin and these items are often fragranced or chemically treated. Some individuals will utilize cloth handkerchiefs and microfiber cloths which have pros such as being reusable, soft, eco-friendly and pocketable. However, these type of items are not single-use can harbor bacteria/allergens, have detergent residue or fabric softeners that can irritate eyes and they are not truly lint-free wherein fibers migrate to the eye. Another common item utilized to manage tears are cosmetic blotting papers and cotton pads. These are thin, portable, precise, blot without rubbing good near makeup. However, these items are designed for facial oil, not eyes and some are infused with powders/fragrances. Further the edges can be stiff and are not sterile, while cotton pads shed fibers and can leave wisps on lashes.

An opportunity exists for a single-use, micro-thin dabbing strip engineered specifically for the eyelid margin something closer to an ocular-safe tear wick than a tissue. Employing a micro-film substrate, an ultra-thin, lint-free biocompatible film, e.g., medical-grade cellulose, PVA, or chitosan-blend with micro-porous channels that wick tears by capillarity without shedding. Utilizing precision geometry by having a narrow, tapered tip with a soft, rounded edge that reaches the meniscus at the lid margin without contacting the cornea. Employing a surface finish that is satin-matte to avoid sticking and is micro-textured for controlled wicking. Developing a device that is skin friendly being hypoallergenic, preservative-free, with rounded corners to prevent micro-abrasions. Eye-watering is common, but utilizing ordinary items such as tissues, napkins, wipes, and cloths while somewhat effective are not ideal. These items shed lint, irritate skin, carry chemicals, and lack the precision needed at the eyelid margin, often making tearing worse or ruining makeup. A convenient micro-filmed dabbing tool purpose-built for tears would address these shortcomings and offer characteristics such as hygienic, ultra-portable, lint-free, makeup-safe, and gentle on skin quietly restoring comfort and confidence in the exact moments when watery eyes are most disruptive.

Accordingly, there is a need for an eye care device operable to manage tears wherein the device includes an upper portion manufactured from a combination of materials such as but not limited to polyamide, polyester and polypropylene wherein the upper portion is supported by a handle member and is operable to absorb tears/moisture from the eye and area proximate thereto.

### SUMMARY OF THE INVENTION

It is the object of the present invention to provide an eye care device operable to assist a user in the capture and absorption of fluid or tears from the eyes wherein the present invention includes a body wherein the body is comprised of a first portion and a second portion.

Another object of the present invention is to provide a fluid or tear absorption device for absorbing fluid and tears secreted from the eye of a user wherein the first portion and second portion of the body are integrally formed.

A further object of the present invention is to provide an eye care device operable to assist a user in the capture and absorption of fluid or tears from the eyes wherein the first portion of the body is planar in manner being generally flat.

Yet a further object of the present invention is to provide a fluid or tear absorption device for absorbing fluid and tears secreted from the eye of a user wherein the second portion of the body is manufactured from an absorbent and wicking material such as a combination of polyester, polyamides and polypropylene.

Still another object of the present invention is to provide a fluid or tear absorption device for absorbing fluid and tears secreted from the eye of a user wherein the second portion of the body includes a distal end that is tapered in form.

An additional object of the present invention is to provide an eye care device operable to assist a user in the capture and absorption of fluid or tears from the eyes wherein the second portion is antibacterial.

Yet a further object of the present invention is to provide a fluid or tear absorption device for absorbing fluid and tears secreted from the eye of a user wherein the device is disposable.

To the accomplishment of the above and related objects the present invention may be embodied in the form illustrated in the accompanying drawings. Attention is called to the fact that the drawings are illustrative only. Variations are contemplated as being a part of the present invention, limited only by the scope of the claims.

### BRIEF DESCRIPTION OF THE DRAWINGS

A more complete understanding of the present invention may be had by reference to the following Detailed Description and appended claims when taken in conjunction with the accompanying Drawings wherein:

FIG. 1 is a side view of the present invention.

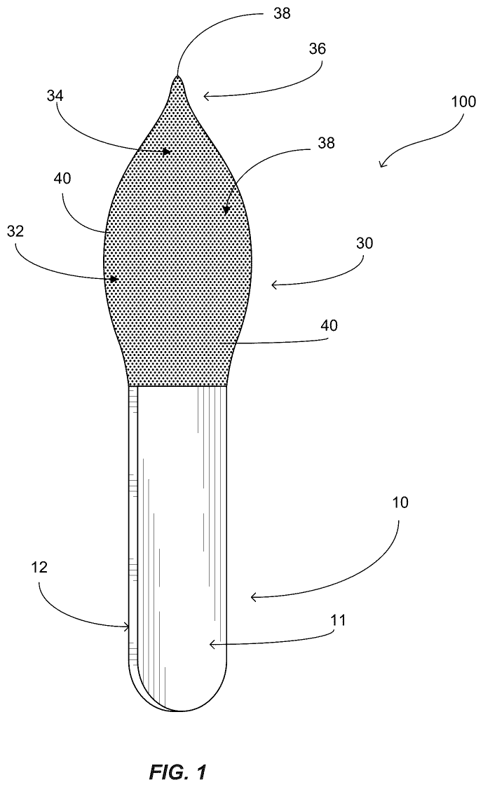

### DETAILED DESCRIPTION

Referring now to the drawings submitted herewith, wherein various elements depicted therein are not necessarily drawn to scale and wherein through the views and figures like elements are referenced with identical reference numerals, there is illustrated an eye care device 100 constructed according to the principles of the present invention.

An embodiment of the present invention is discussed herein with reference to the figures submitted herewith. Those skilled in the art will understand that the detailed description herein with respect to these figures is for explanatory purposes and that it is contemplated within the scope of the present invention that alternative embodiments are plausible. By way of example but not by way of limitation, those having skill in the art in light of the present teachings of the present invention will recognize a plurality of alternate and suitable approaches dependent upon the needs of the particular application to implement the functionality of any given detail described herein, beyond that of the particular implementation choices in the embodiment described herein. Various modifications and embodiments are within the scope of the present invention.

It is to be further understood that the present invention is not limited to the particular methodology, materials, uses and applications described herein, as these may vary. Furthermore, it is also to be understood that the terminology used herein is used for the purpose of describing particular embodiments only, and is not intended to limit the scope of the present invention. It must be noted that as used herein and in the claims, the singular forms “a”, “an” and “the” include the plural reference unless the context clearly dictates otherwise. Thus, for example, a reference to “an element” is a reference to one or more elements and includes equivalents thereof known to those skilled in the art. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

References to “one embodiment”, “an embodiment”, “exemplary embodiments”, and the like may indicate that the embodiment(s) of the invention so described may include a particular feature, structure or characteristic, but not every embodiment necessarily includes the particular feature, structure or characteristic.

Referring in particular to the Figures submitted herewith, the eye care device 100 is comprised of an integrally formed lower portion 10 and upper portion 30 wherein the lower portion 10 and upper portion 30 are integrally formed utilizing suitable durable techniques. The lower portion 10 is planar in manner having a first side 11 and a second side 12. The lower portion 10 further includes a lower end 14 having an arcuate shape thereto. In a preferred embodiment the lower portion 10 is manufactured from a durable rigid material such as but not limited to wood or biodegradable plastic. It should be understood within the scope of the present invention that the lower portion 10 in a preferred embodiment is approximately one to one and a half inches in length. While the preferred embodiment of the lower portion 10 is planar in manner, it is contemplated within the scope of the present invention that the lower portion 10 could be formed in alternate shapes such as but not limited to cylindrical.

The upper portion 30 is manufactured from a lint free absorbent material such as but not limited to a fabric blend of polyamide, polyester and propylene fibers. It is further contemplated within the scope of the present invention that the upper portion 30 could be manufactured from medical grade cellulose. The upper portion 30 is manufactured in a form that provides a bulbous middle section 32 that tapers downward into the integral formation with the lower portion 10. The top section 34 of the upper portion 30 has a precisely formed tip member 36. The tip member 36 features a narrow, tapered tip with a soft, rounded edge 38 that reaches the meniscus at the lid margin of the eye without contacting the cornea. The outer surface 38 of the upper portion 30 is a satin-matte to avoid sticking and is micro-textured for controlled wicking. The outer surface 38 has disposed thereon a multitude of porous channels 40 that wick tears by capillary action directing towards the absorbent middle section 32 that is sized to accommodate a larger volume of liquid.

In the preceding detailed description, reference has been made to the accompanying drawings that form a part hereof, and in which are shown by way of illustration specific embodiments in which the invention may be practiced. These embodiments, and certain variants thereof, have been described in sufficient detail to enable those skilled in the art to practice the invention. It is to be understood that other suitable embodiments may be utilized and that logical changes may be made without departing from the spirit or scope of the invention. The description may omit certain information known to those skilled in the art. The preceding detailed description is, therefore, not intended to be limited to the specific forms set forth herein, but on the contrary, it is intended to cover such alternatives, modifications, and equivalents, as can be reasonably included within the spirit and scope of the appended claims.

What is claimed is:

1. An eye care device comprising:
a lower portion, said lower portion having a first side and a second side, said lower portion being planar in manner, said lower portion being manufactured from a rigid material; and an upper portion, said upper portion being secured to a second end of said lower portion, said upper portion being manufactured from an absorbent material, said upper portion having an outer surface, said upper portion having a middle section that is bulbous in shape, said upper portion having a top section, said top section having a tip member, said tip member having a narrow, tapered tip that reaches the meniscus at the lid margin of the eye.

2. The eye care device as recited in claim 1, wherein the upper portion is manufactured from a combination of polyamide, polyester, polypropylene or medical grade cellulose fibers.

3. The eye care device as recited in claim 2, wherein said outer surface of said upper portion is a satin-matte.

4. The eye care device as recited in claim 2, wherein said outer surface includes a multitude of porous channels operable to wick moisture by capillary action directing towards the middle section.

5. The eye care device as recited in claim 4, wherein said lower portion includes a first end that has an arcuate edge.

6. The eye care device as recited in claim 5, wherein said lower portion is manufactured from wood or biodegradable plastic.
